SEQUENCE LISTING (3 sequences)

SEQ ID NO: 1 - protein, 831 aa, Homo sapiens [sequence omitted]

SEQ ID NO: 2 - protein, 830 aa, Homo sapiens
MERPWGAADGLSRWPHGLGLLLLLQLLPPSTLSQDRLDAPPPPAAPLPRWSGPIGVSWGLRAAAAGGAFPRGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCTDLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAGVLIVKKYVCGGRFLVHRYSVLQQHAEANGVDGVDALDTASHTNKSGYHDDSDEDLLE

SEQ ID NO: 3 - protein, 694 aa, Homo sapiens
MTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCTDLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAGVLIVKKYVCGGRFLVHRYSVLQQHAEANGVDGVDALDTASHTNKSGYHDDSDEDLLE

(19) United States
(12) Patent Application Publication
(10) Pub. No.: US 2024/0132514 A1
(43) Pub. Date: Apr. 25, 2024

(54) PYRIDINE DERIVATIVES AS MODULATORS OF SORTILIN ACTIVITY

(71) Applicant: Insusense ApS, Copenhagen (DK)

(72) Inventors: Paul Brian LITTLE, Copenhagen (DK); Manuel Javier CASES-THOMAS, Copenhagen (DK); Mads Fuglsang KJØLBY, Copenhagen (DK); Anders NYKJÆR, Copenhagen (DK)

(21) Appl. No.: 18/273,225

(22) PCT Filed: Jan. 20, 2022
(86) PCT No.: PCT/EP2022/051282

§ 371 Date: Jul. 19, 2023

(30) Foreign Application Priority Data
Jan. 20, 2021 (EP) 21152638.9

(51) Int. Cl. C07D 491/056; C07D 213/75; C07D 471/04; C07D 491/052

(52) U.S. Cl. CPC C07D 491/056; C07D 213/75; C07D 471/04; C07D 491/052

(57) ABSTRACT

The present invention relates to compounds of formula (I), which are modulators of sortilin activity. The invention also relates to pharmaceutical compositions comprising these compounds and to the use of these compounds in the treatment or prevention of medical conditions where modulation of sortilin activity is beneficial.

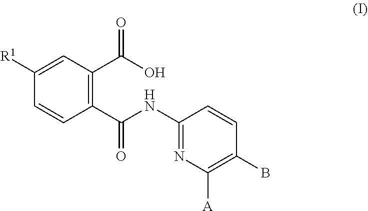

### FIELD OF THE INVENTION

The present invention relates to compounds of formula (I), which are modulators of sortilin activity. The invention also relates to pharmaceutical compositions comprising these compounds and to the use of these compounds in the treatment or prevention of medical conditions where modulation of sortilin activity is beneficial. Such medical conditions include mental diseases, diabetes, diabetes-related conditions and hearing loss.

### BACKGROUND

Sortilin (encoded by SORT1) is a type 1 membrane receptor in the vacuolar protein sorting 10 protein (VPS10P) family of sorting receptors, and is abundantly expressed in the central nervous system, the inner ear, and in some peripheral tissues involved in metabolic control^{1,2,3,4}. Sortilin has an amino acid sequence according to SEQ ID NO: 1 and comprises a signal peptide, a propeptide, the Vps10p domain, a 10cc domain (10CCa+10CCb), a transmembrane domain and a large cytoplasmic tail. The luminal domain of sortilin has 6 potential N-linked glycosylation sites, whilst the cytoplasmic tail enables for the recruitment of various adapter proteins.

Sortilin binds to a vast number of ligands and membrane receptors and as a result engages in functions known to be important in cellular signaling and sorting. For example, sortilin is involved in signaling by proneurotrophins: the proforms of nerve growth factor (proNGF), brain derived neurotrophic factor (proBDNF), and neurotrophin-3 (proNT3), respectively. In complex with the protein p75NTR (p75 neurotrophin receptor), sortilin has been reported to form the receptor for proneurotrophin-mediated apoptotic effects leading to degeneration and cell death in cellular and animal models^{5,6,7}.

Previous work has suggested a role for sortilin in cellular sorting and signalling associated with diseases such as diabetes and obesity (Huang et al 2013 Mol Biol Cell October;24(19):3115-22). Sortilin facilitates translocation of GLUT4 to the plasma membrane and rescues it from degradation in the lysosomes (Pan et al Mol Biol Cell. 2017 June 15;28(12):1667-1675). Sortilin levels have been shown to be modulated by the level of inflammation associated with these diseases. The pro-inflammatory cytokine, TNFα, reduces both mRNA levels and protein levels of sortilin in cultured mouse and human adipocytes, as well as in vivo when injected into mice (Kaddai et al. Diabetologia 52: 932-40, 2009). Sortilin can also influence cytokine secretion: targeting sortilin in immune cells has been proposed to attenuate inflammation and reduce atherosclerosis disease progression (Mortensen et al. J Clin Invest 124(12):5317-22, 2014). Additionally, US 2016/0331746 describes various scaffolds of small molecules capable of binding to the active site of sortilin. Sortilin is involved in the regulation of glucose uptake (Shi & Kandror. Developmental Cell 9:99-108, 2005) and the development of lipid disorder diseases (Gao et al. DNA and Cell Biology 36(12):1050-61, 2017).

Further, plasma sortilin levels have been reported to be a potential biomarker for identifying patients with either coronary heart disease or diabetes mellitus (Oh et al. Cardiovascular Diabetology 16:92, 2017). Patients that showed increased sortilin levels within their plasma, and therefore identifiable as suffering from the above conditions, also displayed enhanced glucose levels suggesting sortilin as a therapeutic target for treating these conditions.

In view of the above, there is an unmet need for new compounds that may be used in the treatment and prevention of medical conditions in which modulation of sortilin is beneficial, such as neurodegenerative disorders, cancer, pain, diabetes mellitus, diabetic retinopathy, cardiovascular disease, hereditary eye conditions and hearing loss.

### DESCRIPTION OF THE FIGURES

FIG. 1 depicts a virtual binding representation showing the interaction between the compound of example 23 and sortilin.

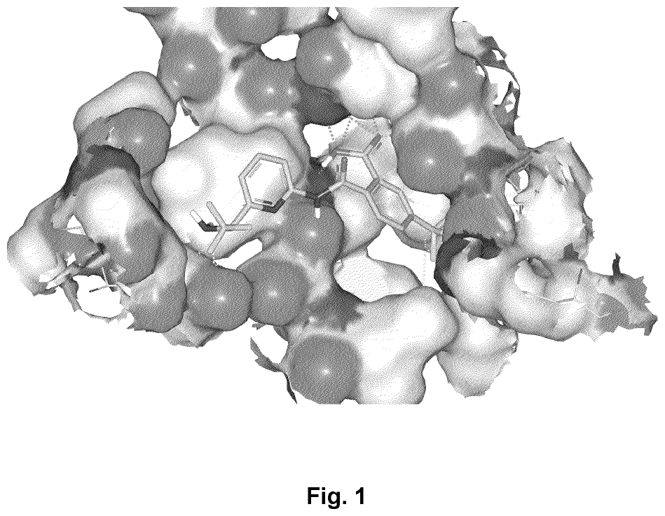

### DISCLOSURE OF THE INVENTION

In a first aspect, the present invention provides a compound of formula (I)

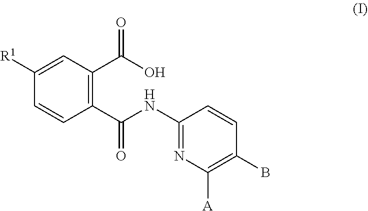

or a pharmaceutically acceptable salt, solvate, hydrate, tautomer, optical isomer, N-oxide, and/or prodrug thereof, wherein
A is selected from the group consisting of H, —OH, C₁-C₄ hydroxyalkyl, C₂-C₄ alkyl, C₁-C₄ haloalkyl, C₁-C₄ alkoxy, C₁-C₄ haloalkoxy and C₁-C₄ aminoalkyl;
B is selected from the group consisting of H, —OH, C₁-C₄ alkyl, C₁-C₄ haloalkyl, C₁-C₄ alkoxy and C₁-C₄ haloalkoxy; or
A and B may be taken together with the carbon atom to which they are respectively attached to form a 5- to 7-membered monocyclic heterocyclic ring that is optionally substituted with one or more substituents independently selected from —OH, C₁-C₄ alkyl, C₁-C₄ haloalkyl and C₁-C₄ alkoxy and C₁-C₄ haloalkoxy; and
R¹ is selected from the group consisting of halo, C₁-C₆ alkyl, C₂-C₆ alkenyl, halo C₁-C₆ alkyl, and halo C₂-C₆ alkenyl.

It has been surprisingly found that compounds of formula (I) inhibit or antagonise sortilin and therefore may be useful in conditions where sortilin inhibition is beneficial. Such conditions include neurodegenerative disorders, cancer, pain, diabetes mellitus, diabetic retinopathy, cardiovascular disease, hereditary eye conditions and hearing loss.

As used herein, the term “sortilin” may refer to full length sortilin (also referred to as immature sortilin), comprising a signal peptide, a propeptide, a Vps10p domain, a 1000 domain, a transmembrane domain and a large cytoplasmic tail, having an amino acid sequence according to SEQ ID NO: 1 or SEQ ID NO: 2, or it may refer to mature sortilin, comprising a Vps10p domain, a 1000 domain, a transmembrane domain and a large cytoplasmic tail, having an amino acid sequence according to SEQ ID NO: 3, ora naturally occurring fragment, homologue or variant thereof. The term “sortilin” or “sortilin molecule” are used interchangeably herein. It is understood that the sortilin is capable of interacting with a pro-neurotrophin molecule to form a sortilin/pro-neurotrophin complex. This sortilin/pro-neurotrophin complex may or may not be capable of interacting with a p75NTR molecule to form a trimeric complex comprising sortilin, pro-neurotrophin and p75NTR. It is understood that this trimeric complex may be responsible for adverse biological responses, such as stimulating apoptosis in retinal and ganglion cells, and controlling growth cone retraction of projecting axons.

As used herein, the term “pro-neurotrophin” refers to the larger precursors of neurotrophins, which undergo proteolytic cleavage to yield the mature form of the neurotrophin. Neurotrophins are a family of proteins that induce the survival, development and function of neurons, and are commonly referred to as growth factors. Pro-neurotrophins are biologically active and have distinct roles compared to their neurotrophin counterparts, such as induction of apoptosis. Examples of pro-neurotrophins include proNGF, proBDNF, proNT3 and proNT4. Pro-neurotrophins may also control synaptic plasticity. Whereas mature neurotrophins induce synaptic strength, in their proforms they may weaken synapses,

The compounds of the invention may be sortilin inhibitors or antagonists. As used herein, the term “sortilin antagonist” refers to a substance that interferes with, blocks, or otherwise attenuates the effect of, a sortilin protein binding to a pro-neurotrophin (e.g., proNGF, proNT3, proBDNF) and preventing the formation of the trimeric complex between sortilin, p75NTR and the pro-neurotrophin. The term “sortilin antagonist” also includes a substance or agent that interferes with the formation of a high affinity trimeric complex. In the latter scenario, it is recognised that a trimeric complex may be formed in that sortilin can bind to p75NTR (but not proNGF) and p75NTR can simultaneously bind the NGF domain of proNGF. However, the resulting trimeric complex may be of lower affinity for its receptor and as a result have significantly reduced capacity to stimulate apoptosis via the mechanism described above. Skeldal et al (J. Biol. Chem. 2012 December 21;287(52):43798-809) demonstrated that the apoptotic function of the trimeric complex is abolished when sortilin is devoid in its intracellular domain. The term “sortilin antagonist” also includes a substance or agent that interferes with, blocks, or otherwise attenuates the effect of, a sortilin protein interacting with p75NTR. This interaction may be completely prevented, in which case the trimeric complex is prevented from forming, or only partially prevented, in which case the trimeric complex may be formed but may have reduced biological potency. Skeldal et al showed that complex formation between sortilin and p75NTR relies on contact points in the extracellular domains of the receptors and that the interaction critically depends on an extracellular juxtamembrane 23-amino acid sequence of p75NTR. Thus, the sortilin antagonist may interfere with this 23-amino acid sequence or proximal sequences in the molecules.

In the first aspect of the invention, it is preferred that R 1 is selected from the group consisting of halo, C₁-C₄ alkyl and halo C₁-C₄ alkyl. It is more preferred that R¹ is fluoro C₁-C₄ alkyl.

In a preferred aspect of the invention, R¹ is selected from the group consisting of CF₃ and t-butyl.

In another preferred aspect of the invention, A is selected from the group consisting of C₁-C₄ hydroxyalkyl and C₁-C₄ alkoxy.

It is also preferred that B is selected from the group consisting of H, C₁-C₄ haloalkyl and C₁-C₄ alkyl. It is more preferred that B is H.

In another preferred aspect of the invention, A and B are taken together with the carbon atom to which they are respectively attached to form a 5- to 7-membered monocyclic heterocyclic ring that is optionally substituted with one or more substituents independently selected from C₁-C₄ alkyl and C₁-C₄ haloalkyl.

It is more preferred that A and B are taken together with the carbon atom to which they are respectively attached to form a 6-membered monocyclic heterocyclic ring that is optionally substituted with one or more substituents independently selected from C₁-C₄ alkyl and C₁-C₄ haloalkyl.

It is more preferred that the 6-membered monocyclic heterocyclic ring is unsubstituted.

It is even more preferred that the 6-membered monocyclic heterocyclic ring comprises an oxygen atom.

In even more preferred embodiments, the compound of formula (I) is a compound of formula (Ia)

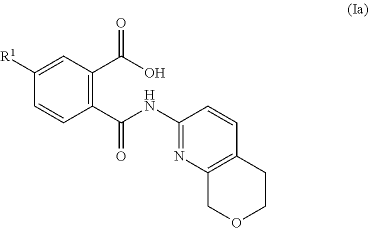

wherein R¹ is selected from the group consisting of halo, C₁-C₄ alkyl and halo C₁-C₄ alkyl. It is more preferred that R¹ is fluoro C₁-C₄ alkyl. It is most preferred that R¹ is selected from the group consisting of CF₃ and t-butyl.

In a particularly preferred embodiment of the invention:
A is selected from the group consisting of C₁-C₄ hydroxyalkyl and C₁-C₄ alkoxy;
B is selected from the group consisting of H, C₁-C₄ haloalkyl and C₁-C₄ alkyl; or
A and B are taken together with the carbon atom to which they are respectively attached to form a 6-membered monocyclic heterocyclic ring that is optionally substituted with one or more substituents independently selected from C₁-C₄ alkyl and C₁-C₄ haloalkyl; and
R¹ is selected from the group consisting of halo, C₁-C₄ alkyl and halo C₁-C₄ alkyl.

In another preferred embodiment of the invention:
A is selected from the group consisting of C₁-C₄ hydroxyalkyl and C₁-C₄ alkoxy;
B is H; or
A and B are taken together with the carbon atom to which they are respectively attached to form an unsubstituted 6-membered monocyclic heterocyclic ring; and
R¹ is selected from the group consisting of CF₃ and t-butyl.

Particular compounds of the invention are those listed below.
2-({5H,6H,8H-pyrano[3,4-b]pyridin-2-yl}carbamoyl)-5-(trifluoromethyl)benzoic acid;
2-{[6-(hydroxymethyl)pyridin-2-yl]carbamoyl}-5-(trifluoromethyl)benzoic acid;
5-tert-butyl-2-{[6-(hydroxymethyl)pyridin-2-yl]carbamoyl}benzoic acid;
(RAC) 2-{[6-(1-hydroxyethyl)pyridin-2-yl]carbamoyl}-5-(trifluoromethyl)benzoic acid;
2-({6-[(1S)-1-hydroxyethyl]pyridin-2-yl}carbamoyl)-5-(trifluoromethyl)benzoic acid;
2-({6-[(1R)-1-hydroxyethyl]pyridin-2-yl}carbamoyl)-5-(trifluoromethyl)benzoic acid;
2-{[6-(2-hydroxypropan-2-yl)pyridin-2-yl]carbamoyl}-5-(trifluoromethyl)benzoic acid;
2-{[6-(2-hydroxyethyl)pyridin-2-yl]carbamoyl}-5-(trifluoromethyl)benzoic acid;
(RAC) 5-tert-butyl-2-{[6-(1-hydroxyethyppyridin-2-yl]carbamoyl}benzoic acid;
5-tert-butyl-2-({6-[(1R)-1-hydroxyethyl]pyridin-2-yl}carbamoyl)benzoic acid;
5-tert-butyl-2-({6-[(1S)-1-hydroxyethyl]pyridin-2-yl}carbamoyl)benzoic acid;
2-({2H,3H,4H-pyrano[2,3-b]pyridin-7-yl}carbamoyl)-5-(trifluoromethyl)benzoic acid;
2-({2H,3H-[1,4]dioxino[2,3-b]pyridin-6-yl}carbamoyl)-5-(trifluoromethyl)benzoic acid;
2-({5H,7H,8H-pyrano[4,3-b]pyridin-2-yl}carbamoyl)-5-(trifluoromethyl)benzoic acid;
2-[(5,6,7,8-tetrahydro-1,6-naphthyridin-2-yl)carbamoyl]-5-(trifluoromethyl)benzoic acid;
5-tert-butyl-2-({5H,6H,8H-pyrano[3,4-b]pyridin-2-yl}carbamoyl)benzoic acid;
(RAC) 2-{[6-(2,2,2-trifluoro-1 -hydroxyethyl)pyridin-2-yl]carbamoyl}-5-(trifluoromethyl)benzoic acid;
2-({6-[(1S)-2,2,2-trifluoro-1-hydroxyethyl]pyridin-2-yl}carbamoyl)-5-(trifluoromethyl)benzoic acid;
2-({6-[(1R)-2,2,2-trifluoro-1-hydroxyethyl]pyridin-2-yl}carbamoyl)-5-(trifluoromethyl)benzoic acid;
(RAC) 2-{[6-(1-amino-2,2,2-trifluoroethyl)pyridin-2-yl]carbamoyl}-5-(trifluoromethyl)benzoic acid;
2-({6-[(1R)-1-amino-2,2,2-trifluoroethyl]pyridin-2-yl}carbamoyl)-5-(trifluoromethyl)benzoic acid;
2-({6-[(1S)-1-amino-2,2,2-trifluoroethyl]pyridin-2-yl}carbamoyl)-5-(trifluoromethyl)benzoic acid;
   or a pharmaceutically acceptable salt, solvate, hydrate, tautomer, optical isomer, N-oxide, and/or prodrug thereof.

The compounds of formula (I) are intended for use in the treatment or prevention of a neurodegenerative disorder, a cancer, pain, diabetes mellitus, diabetic retinopathy, cardiovascular disease, hereditary eye conditions or hearing loss. Preferably, the neurodegenerative disorder is selected from frontotemporal dementia, Alzheimer's disease, Parkinson's disease and spinal cord injury. Preferably, the hearing loss is selected from noise-induced hearing loss, ototoxicity induced hearing loss, age-induced hearing loss, idiopathic hearing loss, tinnitus and sudden hearing loss. Preferably, the cancer is selected from breast cancer, lung cancer, ovarian cancer, prostate cancer, thyroid cancer, pancreatic cancer, glioblastoma, and colorectal cancer. Preferably the cardiovascular disease is selected from atherosclerosis, cardiomyopathy, heart attack, arrhythmias, and coronary artery disease.

Thus, in an embodiment, the compounds for use according to the invention may disrupt interaction between a sortilin molecule and a pro-neurotrophin molecule, or disrupt the interaction between a sortilin molecule and a p75NTR molecule. Said sortilin molecule may be mature sortilin.

Preferably, the compounds of the present invention are sortilin inhibitors. As used herein, the term “sortilin inhibitor” refers to a compound that binds to a sortilin protein, thereby preventing it from binding to a pro-neurotrophin or a p75NTR molecule and preventing the formation of the aforementioned trimeric complex, or resulting in the formation of a trimeric complex that is less active or inactive.

Preferably, the compounds of the present invention prevent the protein-protein interaction between a sortilin molecule and a pro-neurotrophin or a p75NTR molecule, further preventing the formation of the apoptotic trimeric complex usually formed between sortilin, pro-neurotrophin and the p75NTR receptor, or resulting in the formation of a low affinity trimeric complex, which is biologically less active or inactive or has minimal activity.

Thus, the compound may bind to sortilin, a pro-neutrophin or a p75NTR molecule. The antagonistic action may be due to direct blocking of protein-protein interaction or it could be by steric hindrance when bound at a site of one of these proteins apart from the binding site.

According to a second aspect of the invention, there is provided a pharmaceutical composition comprising a compound according to the first aspect of the invention and one or more pharmaceutically acceptable carriers, excipients, and/or diluents.

In a third aspect of the invention, there is provided a compound according to the first aspect of the invention, or a pharmaceutical composition according to the second aspect of the invention for use in therapy.

According to a fourth aspect of the invention, there is provided a compound according to the first aspect of the invention, or a pharmaceutical composition according to the second aspect of the invention for use in the treatment or prevention of a neurodegenerative disorder, a cancer, pain, diabetes mellitus, diabetic retinopathy, cardiovascular disease, hereditary eye conditions or hearing loss.

Preferably, the neurodegenerative disorder is selected from frontotemporal dementia, Alzheimer's disease, Parkinson's disease and spinal cord injury.

Preferably, the hearing loss is selected from noise-induced hearing loss, ototoxicity induced hearing loss, age-induced hearing loss, idiopathic hearing loss, tinnitus and sudden hearing loss.

Preferably, the cancer is selected from breast cancer, lung cancer, ovarian cancer, prostate cancer, thyroid cancer, pancreatic cancer, glioblastoma, and colorectal cancer.

Preferably the cardiovascular disease is selected from atherosclerosis, cardiomyopathy, heard attack, arrhythmias, and coronary artery disease.

According to a fifth aspect of the invention, there is provided the use of the compound according to the first aspect of the invention for the manufacture of a medicament for the treatment or prevention of a neurodegenerative disorder, a cancer, pain, diabetes mellitus, diabetic retinopathy, cardiovascular disease, hereditary eye conditions or hearing loss.

According to a sixth aspect of the invention, there is provided a method for the treatment or prevention of a disease or condition responsive to sortilin modulation comprising administering a therapeutically effective amount of the compound according to the first aspect of the invention or the pharmaceutical composition according the second aspect of the invention.

The compounds of the invention may include isotopically-labelled and/or isotopically-enriched forms of the compounds. The compounds of the invention herein may contain unnatural proportions of atomic isotopes at one or more of the atoms that constitute such compounds. Examples of isotopes that can be incorporated into the disclosed compounds include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F , ³⁶Cl.

The compounds of the invention may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Compounds that have acidic properties can be converted to their pharmaceutically acceptable basic addition salts by treating the acid form with an appropriate base. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

Throughout the present disclosure, a given chemical formula or name shall also encompass all pharmaceutically acceptable salts, solvates, hydrates, N-oxides, and/or prodrug forms thereof. It is to be understood that the compounds of the invention include any and all hydrates and/or solvates of the compound formulas. It is appreciated that certain functional groups, such as the hydroxy, amino, and like groups form complexes and/or coordination compounds with water and/or various solvents, in the various physical forms of the compounds. Accordingly, the above formulas are to be understood to include and represent those various hydrates and/or solvates.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H, 2H- and 4H- 1,2,4-triazole, 1H- and 2H- isoindole, and 1H- and 2H-pyrazole. Tautomeric forms can be in equilibrium or sterically locked into one form by appropriate substitution.

The compounds described herein can be asymmetric (e.g. having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis- and trans-geometric isomers of the compounds of the present invention are described and may be isolated as a mixture of isomers or as separated isomeric forms.

In the case of the compounds which contain an asymmetric carbon atom, the invention relates to the D form, the L form, and D,L mixtures and also, where more than one asymmetric carbon atom is present, to the diastereomeric forms. Those compounds of the invention which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

The term “prodrugs” refers to compounds that may be converted under physiological conditions or by solvolysis to a biologically active compound of the invention. A prodrug may be inactive when administered to a subject in need thereof, but is converted in vivo to an active compound of the invention. Prodrugs are typically rapidly transformed in vivo to yield the parent compound of the invention, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2nd Ed., Elsevier Academic Press (2004), page 498 to 549). Prodrugs of a compound of the invention may be prepared by modifying functional groups, such as a hydroxy, amino or mercapto groups, present in a compound of the invention in such a way that the modifications are cleaved, either in routine manipulation or in vivo, to the parent compound of the invention. Examples of prodrugs include, but are not limited to, acetate, formate and succinate derivatives of hydroxy functional groups or phenyl carbamate derivatives of amino functional groups.

The term “treatment” as used herein may include prophylaxis of the named disorder or condition, or amelioration or elimination of the disorder once it has been established. The term “prevention” refers to prophylaxis of the named disorder or condition.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic imaging or sampling of subject tissue, fluids, specimens, cells, proteins, chemical markers, genetic materials, etc. as markers or indicators of the treatment regimen. In other methods, the subject is pre-screened or identified as in need of such treatment by assessment for a relevant marker or indicator of suitability for such treatment.

The invention provides a method of monitoring treatment progress. The method includes the step of determining a level of diagnostic marker (Marker) (e.g. any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pretreatment level of Marker can then be compared to the level of Marker in the subject after the treatment commences, to determine the efficacy of the treatment.

A level of Marker or Marker activity in a subject may be determined at least once. Comparison of Marker levels, e.g., to another measurement of Marker level obtained previously or subsequently from the same patient, another patient, or a normal subject, may be useful in determining whether therapy according to the invention is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, is ELISA, radiolabeling/assay techniques, blotting/chemiluminescence methods, real-time PCR, and the like.

For clinical use, the compounds disclosed herein are formulated into pharmaceutical compositions (or formulations) for various modes of administration. It will be appreciated that compounds of the invention may be administered together with a physiologically acceptable carrier, excipient, and/or diluent (i.e. one, two, or all three of these). The pharmaceutical compositions disclosed herein may be administered by any suitable route, preferably by oral, rectal, nasal, topical (including ophthalmic, buccal and sublingual), sublingual, transdermal, intrathecal, transmucosal or parenteral (including subcutaneous, intramuscular, intravenous and intradermal) administration. Other formulations may conveniently be presented in unit dosage form, e.g., tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration. The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds disclosed herein may be incorporated into slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

### DEFINITIONS

“Optional” or “optionally” means that the subsequently described event or circumstance may, but need not, occur, and that the description includes instances where the event or circumstance occurs and instances in which it does not.

The term “heteroatom” means O, N, or S.

The term “C₁-C₄ alkyl” denotes a straight, branched or cyclic or partially cyclic alkyl group having from 1 to 4 carbon atoms, i.e. 1, 2, 3 or 4 carbon atoms. For the “C₁-C₄ alkyl” group to comprise a cyclic portion it should be formed of 3 to 4 carbon atoms. For parts of the range “C₁-C₄ alkyl” all subgroups thereof are contemplated, such as C₁-C₄ alkyl, C₁-C₃ alkyl, C₁-C₂ alkyl, C₁ alkyl, C₂-C₄ alkyl, C₂-C₃ alkyl, C₂ alkyl, C₃-C₄ alkyl, C₃ alkyl and C₄ alkyl. Examples of “C₁-C₄ alkyl” include methyl, ethyl, n-propyl, isopropyl, cyclopropyl, n-butyl, isobutyl, sec-butyl, t-butyl, cyclobutyl and cyclopropylmethyl.

The term “C₁-C₄ hydroxyalkyl” denotes a C₁-C₄ alkyl as described above substituted with an —OH group. Examples of “C₁-C₄ hydroxyalkyl” include hydroxymethyl, 1-hydroxyethyl, 2-hydroxyethyl, 1-hydroxypropyl, 2-hydroxypropyl and 3-hydroxypropyl.

The term “C₁-C₄ aminoalkyl” denotes a C₁-C₄ alkyl as described above substituted with an —NH₂ group.

The term “C₁-C₄ haloalkyl” denotes a C₁-C₄ alkyl as described above substituted with a halogen atom, which is preferably, F, Cl, Br and I, more preferably F and Cl, and most preferably F.

The term “C₁-C₄ alkyl” denotes a straight, branched or cyclic or partially cyclic alkyl group having from 1 to 6 carbon atoms, i.e. 1, 2, 3, 4, 5 or 6 carbon atoms. For the “C₁-C₆ alkyl” group to comprise a cyclic portion it should be formed of 3 to 6 carbon atoms. For parts of the range “C₁-C₆ alkyl” all subgroups thereof are contemplated, such as C₁-C₅ alkyl, C₁-C₄ alkyl, C₁-C₃ alkyl, C₁-C₂ alkyl, C₁ alkyl, C₂-C₆ alkyl, C₂-C₅ alkyl, C₂-C₄ alkyl, C₂-C₃ alkyl, C₂ alkyl, C₃-C₆ alkyl, C₃-C₅ alkyl, C₃-C₄ alkyl, C₃ alkyl, C₄-C₆ alkyl, C₄-C₅ alkyl, C₄ alkyl, C₅ -C₆ alkyl, C₅ alkyl, and C₆ alkyl. Examples of “C₁-C₆ alkyl” include methyl, ethyl, n-propyl, isopropyl, cyclopropyl, n-butyl, isobutyl, sec-butyl, t-butyl, cyclobutyl, cyclopropylmethyl, and straight, branched or cyclic or partially cyclic pentyl and hexyl etc.

When a term denotes a range, for instance “1 to 6 carbon atoms” in the definition of C₁-C₆ alkyl, each integer is considered to be disclosed, i.e. 1, 2, 3, 4, 5 and 6.

The term “C₂-C₆ alkenyl” denotes a straight, branched or cyclic or partially cyclic alkyl group having at least one carbon-carbon double bond, and having from 2 to 6 carbon atoms. The alkenyl group may comprise a ring formed of 3 to 6 carbon atoms. For parts of the range “C₂-C₆ alkenyl” all subgroups thereof are contemplated, such as C₂-C₅ alkenyl, C₂-C₅ alkenyl, C₂-C₃ alkenyl, C₂ alkenyl, C₃-C₆ alkenyl, C₃-C₅ alkenyl, C₃-C₄ alkenyl, C₃ alkenyl, C₄-C₆ alkenyl, C₄-C₅ alkenyl, C₄ alkenyl, C₅-C₆ alkenyl, C₅ alkenyl, and C₆ alkenyl. Examples of “C₂-C₆ alkenyl” include 2-propenyl, 2-butenyl, 3-butenyl, 2-methyl-2-propenyl, 2-hexenyl, 5-hexenyl, 2,3-dimethyl-2-butenyl.

The term “C₁-C₄ alkoxy” denotes —O—(C₁-C₄alkyl) in which a C₁-C₄ alkyl group is as defined above and is attached to the remainder of the compound through an oxygen atom. Examples of “C₁-C₄ alkoxy” include methoxy, ethoxy, n-propoxy, isopropoxy, n-butoxy, isobutoxy, sec-butoxy and t-butoxy.

The term “C₁-C₄ haloalkoxy” denotes a C₁-C₄ alkoxy as described above substituted with a halogen atom, which is preferably, F, Cl, Br and I, more preferably F and Cl, and most preferably F.

The term “halo” means a halogen atom, and is preferably, F, Cl, Br and I, more preferably F and Cl, and most preferably F.

The term “5- to 7-membered monocyclic heterocyclic ring” denotes a non-aromatic monocyclic ring system having 5 to 7 ring atoms, in which at least one ring atoms is a heteroatom.

“An effective amount” refers to an amount of a compound of the invention that confers a therapeutic effect on the treated subject. The therapeutic effect may be objective (i.e. measurable by some test or marker) or subjective (i.e. subject gives an indication of or feels an effect).

As used herein, the terms “administration” or “administering” mean a route of administration for a compound disclosed herein. Exemplary routes of administration include, but are not limited to, oral, intraocular, intravenous, intraperitoneal, intraarterial, and intramuscular. The preferred route of administration can vary depending on various factors, e.g. the components of the pharmaceutical composition comprising a compound disclosed herein, site of the potential or actual disease and severity of disease.

The terms “subject” and “patient” are used herein interchangeably. They refer to a human or another mammal (e.g., mouse, rat, rabbit, dog, cat, cattle, swine, sheep, horse or primate) that can be afflicted with or is susceptible to a disease or disorder but may or may not have the disease or disorder. It is preferred that the subject is human.

Compounds of the invention may be disclosed by the name or chemical structure. If a discrepancy exists between the name of a compound and its associated chemical structure, then the chemical structure prevails.

The invention will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilise the present invention to its fullest extent. All references and publications cited herein are hereby incorporated by reference in their entirety.

### PREPARATION OF COMPOUNDS OF THE INVENTION

The compounds of formula (I) disclosed herein may be prepared by, or in analogy with, conventional methods. Appropriate reaction conditions for the individual reaction steps are known to a person skilled in the art. The necessary starting materials for preparing the compounds of formula (I) are either commercially available, or may be prepared by methods known in the art.

The compounds of formula (I) may possess one or more chiral carbon atoms, and they may therefore be obtained in the form of optical isomers, e.g., as a pure enantiomer, or as a mixture of enantiomers or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

Particular experimental procedures for examples of the invention are described below. The processes may be carried out to give a compound of the invention in the form of a neutral molecule, for example as a protonated carboxylic acid or free base, or as an ionic molecule with a basic or acid addition salt respectively. A pharmaceutically acceptable basic or acid addition salt may be obtained by dissolving the free base in a suitable organic solvent and treating the solution with a base or an acid, in accordance with conventional procedures for preparing basic and acid addition salts from acid or base compounds. Examples of addition salt forming acids are mentioned above.

The chemicals used in the synthetic routes delineated herein may include, for example, solvents, reagents, catalysts, and protecting group and deprotecting group reagents. Examples of protecting groups are t-butoxycarbonyl (Boc), benzyl and trityl(triphenylmethyl). The methods described below may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3rd Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The invention will now be further illustrated by the following non-limiting examples. The specific examples below are to be construed as merely illustrative, and not !imitative of the remainder of the disclosure in any way whatsoever. Without further elaboration, it is believed that one skilled in the art can, based on the description herein, utilize the present invention to its fullest extent. All references and publications cited herein are hereby incorporated by reference in their entirety.

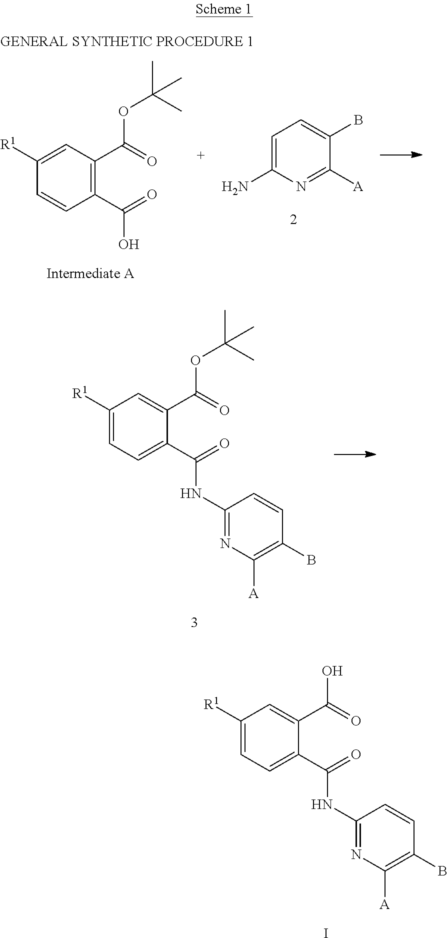

In general synthetic procedure 1, the free carboxylic acid present in Intermediate A is coupled with a suitably substituted amino pyridine using standard amide coupling techniques, for example by mixing a compound like Intermediate A and a suitable 2-aminopyridine 2 in the presence a coupling agent such as HATU and a nitrogen containing amine, such as DIPEA in a suitable organic solvent, such as DMF, at room temperature to afford an intermediate of general formula 3. The compounds of general formula I can, then, be obtained from 3, by deprotection of the tent-butyl ester group using, for example, neat formic acid.

Intermediate A, can be obtained, as shown in Scheme 2, with the required, regiochemical substitution pattern, from commercially available compounds of general structure 4, 5 or 6. When starting from intermediates of general structure 4, the carboxylic acid can be activated with a chlorinating reagent, for example thionyl chloride, in a suitable solvent such as dichloromethane. This provides intermediates of general structure 5 that upon treatment with a metal salt of tent-butanol, such as sodium or potassium tert-butanoate in a suitable solvent such as tent-butanol, to afford intermediate 6. Benzylic oxidation of the methyl substituent to the carboxylic acid general structure as shown in Intermediate A, can be achieved, for example, by chemoselective bromination to give general intermediate 7, using a brominating reagent like for example N-bromosuccinimide. The benzyl bromide, in 7, can yield the aldehyde containing intermediate of general structure 8, by reaction with an oxidizing reagent, for example N-methylmorpholine N-oxide, (NMO). Intermediate A can be then obtained by a secondary oxidation of the aldehyde functionality present in intermediate of general formula 8 with the appropriate oxidizing system, for illustration by using a mixture of sodium chlorite, sodium dihydrogen phosphate and 2-methyl-2-butene in a suitable solvent like, for example, a mixture of t-butanol and water.

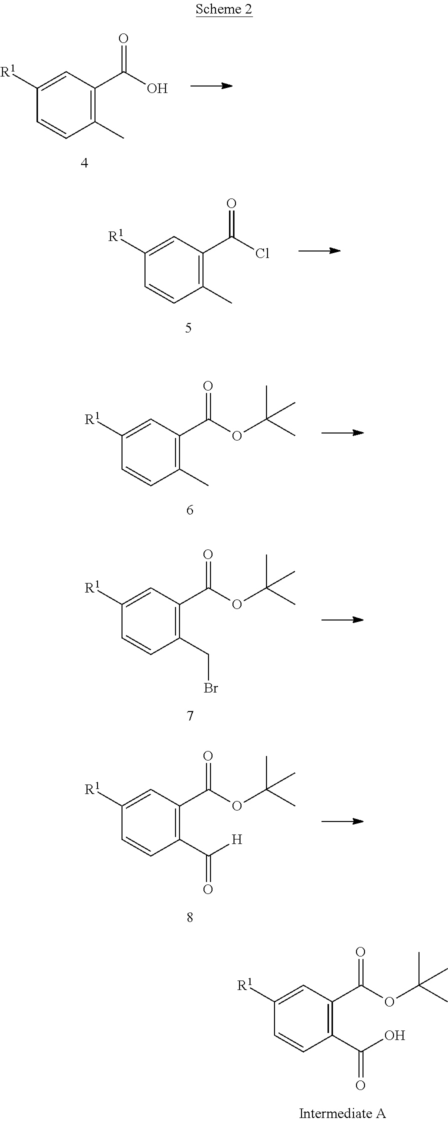

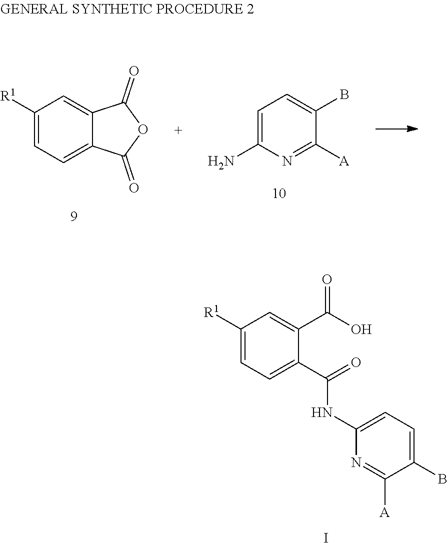

In general synthetic procedure 2, a phthalic anhydride of general structure 9 is reacted with a suitably substituted 2-aminopyridine followed by separation of the two regioisomeric amides by chromatography, for example by using supercritical fluid chromatography (SFC), to produce the desired compound of general structure I.

### EXAMPLES

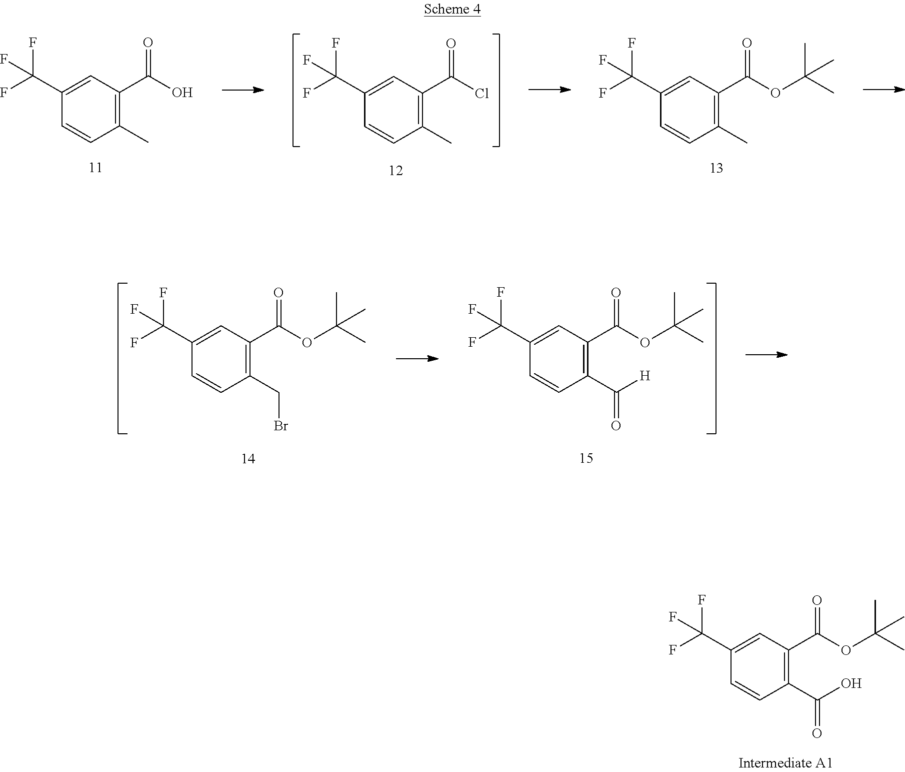

### Intermediate 1A 2-(tert-butoxycarbonyl)-4-(trifluoromethyl) benzoic acid

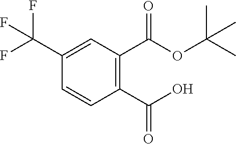 [SMILES: CC(C)(C)OC(=O)c1cc(C(F)(F)F)ccc1C(=O)O]

### Synthesised According to Scheme 4.

Oxalyl dichloride (3.73 g, 2.52 mL, 1.2 Eq, 29.4 mmol) was added to a stirred suspension of 2-methyl-5-(trifluoromethyl)benzoic acid (5.00 g, 1 Eq, 24.5 mmol) and N,N-dimethylformamide (35.8 mg, 37.9 μL, 0.02 Eq, 490 pmol) in anhydrous dichloromethane (100 ml) at room temperature under nitrogen. Vigorous effervescence was observed and after 10 minutes all solid had dissolved. After 2 hours effervescence had stopped and tent-butanol (9.08 g, 11.7 mL, 5 Eq, 122 mmol) followed by pyridine (2.32 g, 2.38 mL, 1.2 Eq, 29.4 mmol) were added. The resulting solution was stirred at room temperature for 18 hours then heated to 35° C. for 4 days. Most of the dichloromethane was evaporated in vacuo. The mixture was diluted with TBME (100 ml), washed with 1M hydrochloric acid (50 ml) then sodium bicarbonate solution (50 ml) then brine (25 ml), dried (MgSO₄) and evaporated in vacuo to a crude solid 13 (5.001 g), ¹H NMR was consistent with a mixture of tent-butyl ester and dimer acid anhydride. The solid was dissolved in TBME (100 ml) and N,N-dimethylethylenediamine (1.00g) was added. After 30 minutes, complete conversion of the anhydride to the corresponding ester and carboxylic acid was achieved. The solution was washed with 1M hydrochloric acid (50 ml), sodium bicarbonate solution (50 ml), brine (25 ml), dried (MgSO₄) and evaporated in vacuo to an oil to yield intermediate 13 (3.31g)

¹H NMR in DMSO-d: (400 MHz, DMSO-d6) δ7.96 (d, J=2.1 Hz, 1H), 7.80 (dd, J=8.1, 2.1 Hz, 1H), 7.55 (d, J=8.0 Hz, 1H), 2.56 (s, 3H), 1.55 (s, 9H).

N-bromosuccinimide (2.46 g, 1.20 Eq, 13.8 mmol) was added to a stirred solution of tert-butyl 2-methyl-5-(trifluoromethyl)benzoate 13 (3.16 g, 95% Wt, 1.00 Eq, 11.5 mmol) in Chloroform (60 mL). The mixture was heated to reflux and AIBN (94.7 mg, 0.05 Eq, 577 μmol) was added. After 30 minutes there was evidence of reaction by thin layer chromatography (silica plate,9/1 hexane/ethyl acetate) indicating formation of a close-running product. The mixture was cooled to room temperature and the chloroform removed in vacuo. The residue was redissolved in anhydrous Acetonitrile (60 mL) and 4-methylmorpholine 4-oxide (2.70 g, 2.00 Eq, 23.1 mmol) added. The mixture was stirred at room temperature for 20 hours. The mixture was diluted with TBME (120 ml), washed with 1M hydrochloric acid (60 ml) then 1M sodium hydroxide solution (60 ml) then brine (60 ml), dried (MgSO₄) and evaporated in vacuo to intermediate 15 as an oil (2.83g) ¹H NMR in DMSO-d6 was consistent with product structure at estimated purity ca. 25% purity. This material was taken directly to next stage without further purification.

Sodium chlorite (467 mg, 2.00 Eq, 5.16 mmol) was added to a stirred solution of crude tert-butyl 2-formyl-5-(trifluoromethyl) benzoate (2.83 g, 25% Wt, 1.00 Eq, 2.58 mmol), sodium dihydrogen phosphate (310 mg, 1.00 Eq, 2.58 mmol) and 2-methyl-2-butene (905 mg, 1.37 mL, 5.00 Eq, 12.9 mmol) in a mixture of t-butanol (4 mL) and water (1 mL). The reaction mixture was stirred for 18 hours at room temperature then extra sodium chlorite (467 mg, 2.00 Eq, 5.16 mmol) was added and stirring continued for 2 hours whereupon reaction was complete. The mixture was diluted with ethyl acetate (50 ml), washed with a mixture of 1M hydrochloric acid (40 ml) and sodium metabisulphite solution (10 ml), dried (MgSO₄) and evaporated in vacuo. The residue was subjected to column chromatography (40 g Flash Pure silica cartridge) with liquid loading in dichloromethane/isohexane (1:2, 6 ml) followed by elution with a 0-50% gradient of 99/1 ethyl acetate/acetic acid in isohexane. Fractions containing the desired product were evaporated in vacuo and the residue co-evaporated with toluene (10 ml) to give Intermediate A1 an oil that solidified (0.638 g, 2.1mmol, 81%, 95% Purity).

### Preparation of Example 1

### 2-((5,8-dihydro-6H-pyrano[3,4-b]pyridin-2-yl)carbamoyl)-5-(trifluoromethyl)benzoic acid

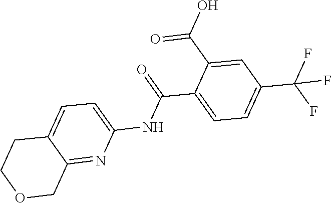 [SMILES: O=C(O)c1cc(C(F)(F)F)ccc1C(=O)Nc1ccc2c(n1)COCC2]

### Intermediate 17

### Tert-butyl 2-({5H,6H,8H-pyrano[3,4-b]pyridin-2-yl}carbamoyl)-5-(trifluoromethyl)benzoate

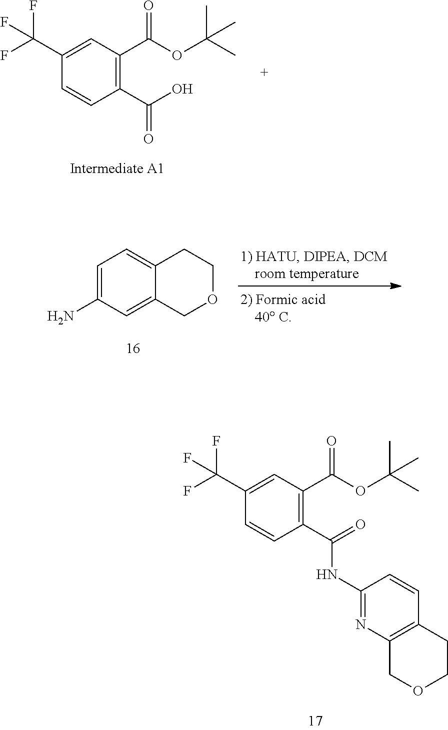 [SMILES: CC(C)(C)OC(=O)c1cc(C(F)(F)F)ccc1C(=O)O ; Nc1ccc2c(c1)COCC2 ; CC(C)(C)OC(=O)c1cc(C(F)(F)F)ccc1C(=O)Nc1ccc2c(n1)COCC2]

HATU (72.1 mg, 0.19 mmol, 1.1 equiv.) was added to a mixture of 2-(tert-butoxycarbonyl)-4-(trifluoromethyl)benzoic acid (50 mg, 0.17 mmol), 5,8-dihydro-6H-pyrano[3,4-b]pyridin-2-amine (28.5 mg, 0.19 mmol, 1 equiv.) and diisopropylethylamine (90 μl, 0.52 mmol, 3 equiv.) in dichloromethane (1 mL) was added and the mixture was stirred for 16 hours at room temperature.

Formic acid (2 mL) was added and the mixture was stirred at 40° C. for 24 hours. The solvent was evaporated in vacuo and residue was taken up in DMSO (1 mL) and purified by preparative HPLC, (Method PREP_ACID-AS4A). The product containing fractions were combined and lyophilised resulting in 14 mg (0.04 mmol, 11% of theory) of the title compound.

LCMS (Method 3: UPLC_AN_BASE, 0.836 min; M+H=367.2; calculated. 367.1)

¹H NMR (400 MHz, DMSO) δ13.54 (s, 1H), 10.99 (s, 1H), 8.12 (d, J=1.9 Hz, 1H), 8.01 (d, J=7.8 Hz, 2H), 7.74 (d, J=8.0 Hz, 1H), 7.63 (d, J=8.4 Hz, 1H), 4.58 (s, 2H), 3.90 (t, J=5.6 Hz, 2H), 2.83-2.76 (m, 2H).

### Examples 2 to 23

Examples 2 to 23 depicted in Table 1, can be prepared in accordance with general synthetic procedures 1 and/or 2.

TABLE 1

| Ex. | Compound Name | Structure |
|---|---|---|
| 2 | 2-({5H,6H,8H-pyrano[3,4- b]pyridin-2-yl}carbamoyl)-5- (trifluoromethyl)benzoic acid | 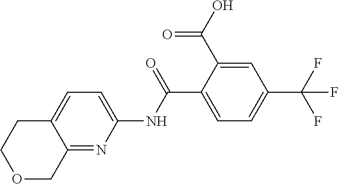 [SMILES: O=C(O)c1cc(C(F)(F)F)ccc1C(=O)Nc1ccc2c(n1)COCC2] |
| 3 | 2-{[6- (hydroxymethyl)pyridin-2- yl]carbamoyl}-5- (trifluoromethyl)benzoic acid | 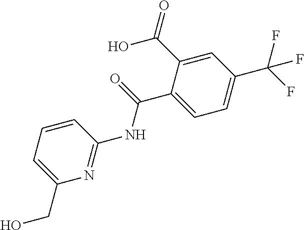 [SMILES: O=C(O)c1cc(C(F)(F)F)ccc1C(=O)Nc1cccc(CO)n1] |
| 4 | 5-tert-butyl-2-{[6- (hydroxymethyl)pyridin-2- yl]carbamoyl}benzoic acid | 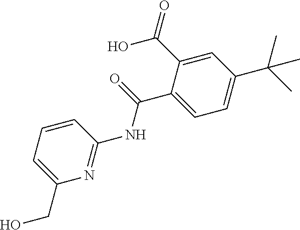 [SMILES: CC(C)(C)c1ccc(C(=O)Nc2cccc(CO)n2)c(C(=O)O)c1] |
| 5 | (RAC) 2-{[6-(1- hydroxyethyl)pyridin-2- yl]carbamoyl}-5- (trifluoromethyl)benzoic acid | 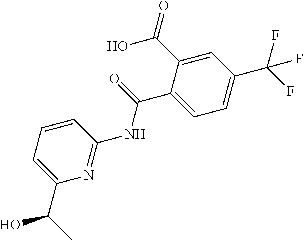 [SMILES: C[C@@H](O)c1cccc(NC(=O)c2ccc(C(F)(F)F)cc2C(=O)O)n1] |
| 6 | 2-({6-[(1S)-1- hydroxyethyl]pyridin-2- yl}carbamoyl)-5- (trifluoromethyl)benzoic acid | 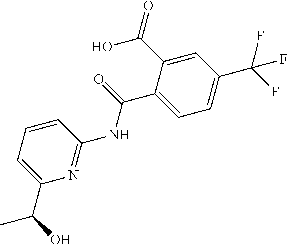 [SMILES: C[C@H](O)c1cccc(NC(=O)c2ccc(C(F)(F)F)cc2C(=O)O)n1] |
| 7 | 2-({6-[(1R)-1- hydroxyethyl]pyridin-2- yl}carbamoyl)-5- (trifluoromethyl)benzoic acid | 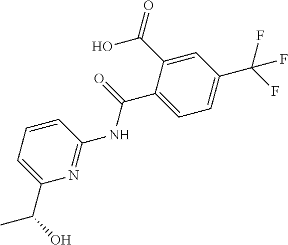 [SMILES: C[C@@H](O)c1cccc(NC(=O)c2ccc(C(F)(F)F)cc2C(=O)O)n1] |
| 8 | 2-{[6-(2-hydroxypropan-2- yl)pyridin-2-yl]carbamoyl}-5- (trifluoromethyl)benzoic acid | 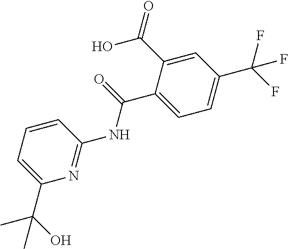 [SMILES: CC(C)(O)c1cccc(NC(=O)c2ccc(C(F)(F)F)cc2C(=O)O)n1] |
| 9 | 2-{[6-(2- hydroxyethyl)pyridin-2- yl]carbamoyl}-5- (trifluoromethyl)benzoic acid | 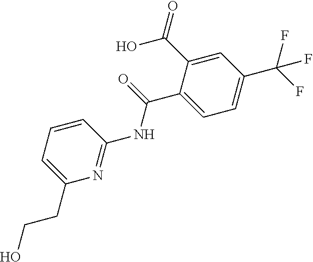 [SMILES: O=C(O)c1cc(C(F)(F)F)ccc1C(=O)Nc1cccc(CCO)n1] |
| 10 | (RAC) 5-tert-butyl-2-{[6-(1- hydroxyethyl)pyridin-2- yl]carbamoyl}benzoic acid | 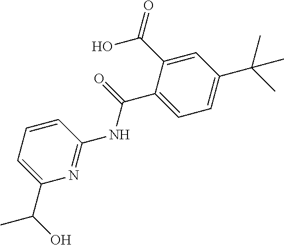 [SMILES: CC(O)c1cccc(NC(=O)c2ccc(C(C)(C)C)cc2C(=O)O)n1] |
| 11 | 5-tert-butyl-2-({6-[(1R)-1- hydroxyethyl]pyridin-2- yl}carbamoyl)benzoic acid | 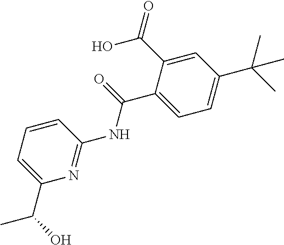 [SMILES: C[C@@H](O)c1cccc(NC(=O)c2ccc(C(C)(C)C)cc2C(=O)O)n1] |
| 12 | 5-tert-butyl-2-({6-[(1S)-1- hydroxyethyl]pyridin-2- yl}carbamoyl)benzoic acid | 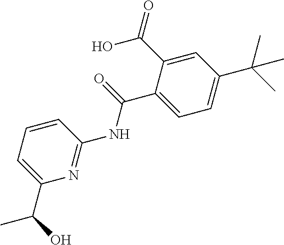 [SMILES: C[C@H](O)c1cccc(NC(=O)c2ccc(C(C)(C)C)cc2C(=O)O)n1] |
| 13 | 2-({2H,3H,4H-pyrano[2,3- b]pyridin-7-yl}carbamoyl)-5- (trifluoromethyl)benzoic acid | 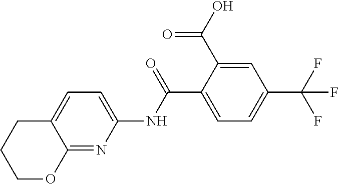 [SMILES: O=C(O)c1cc(C(F)(F)F)ccc1C(=O)Nc1ccc2c(n1)OCCC2] |
| 14 | 2-({2H,3H-[1,4]dioxino[2,3- b]pyridin-6-yl}carbamoyl)-5- (trifluoromethyl)benzoic acid | 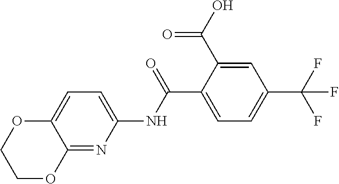 [SMILES: O=C(O)c1cc(C(F)(F)F)ccc1C(=O)Nc1ccc2c(n1)OCCO2] |
| 15 | 2-({5H,7H,8H-pyrano[4,3- b]pyridin-2-yl}carbamoyl)-5- (trifluoromethyl)benzoic acid | 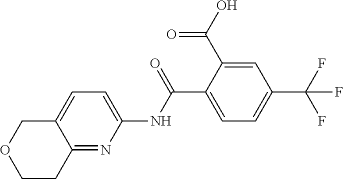 [SMILES: O=C(O)c1cc(C(F)(F)F)ccc1C(=O)Nc1ccc2c(n1)CCOC2] |
| 16 | 2-[(5,6,7,8-tetrahydro-1,6- naphthyridin-2- yl)carbamoyl]-5- (trifluoromethyl)benzoic acid | 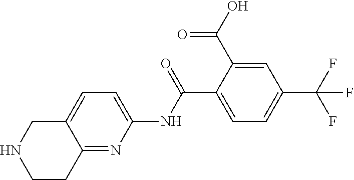 [SMILES: O=C(O)c1cc(C(F)(F)F)ccc1C(=O)Nc1ccc2c(n1)CCNC2] |
| 17 | 5-tert-butyl-2-({5H,6H,8H- pyrano[3,4-b]pyridin-2- yl}carbamoyl)benzoic acid | 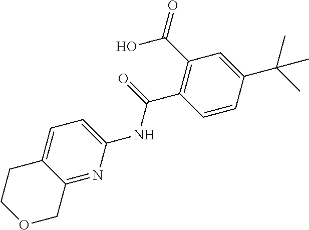 [SMILES: CC(C)(C)c1ccc(C(=O)Nc2ccc3c(n2)COCC3)c(C(=O)O)c1] |
| 18 | (RAC) 2-{[6-(2,2,2-trifluoro- 1-hydroxyethyl)pyridin-2- yl]carbamoyl}-5- (trifluoromethyl)benzoic acid | 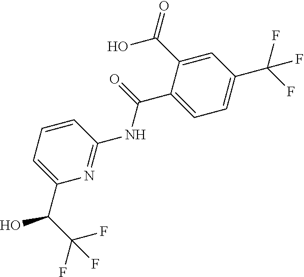 [SMILES: O=C(O)c1cc(C(F)(F)F)ccc1C(=O)Nc1cccc([C@H](O)C(F)(F)F)n1] |
| 19 | 2-({6-[(1S)-2,2,2-trifluoro-1- hydroxyethyl]pyridin-2- yl}carbamoyl)-5- (trifluoromethyl)benzoic acid | 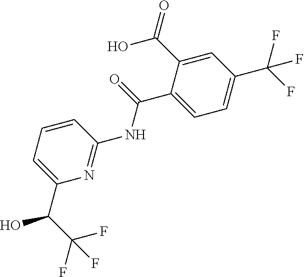 [SMILES: O=C(O)c1cc(C(F)(F)F)ccc1C(=O)Nc1cccc([C@H](O)C(F)(F)F)n1] |
| 20 | 2-({6-[(1R)-2,2,2-trifluoro-1- hydroxyethyl]pyridin-2- yl}carbamoyl)-5- (trifluoromethyl)benzoic acid | 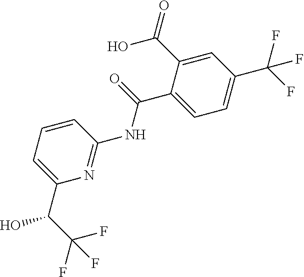 [SMILES: O=C(O)c1cc(C(F)(F)F)ccc1C(=O)Nc1cccc([C@@H](O)C(F)(F)F)n1] |
| 21 | (RAC) 2-{[6-(1-amino-2,2,2- trifluoroethyl)pyridin-2- yl]carbamoyl}-5- (trifluoromethyl)benzoic acid | 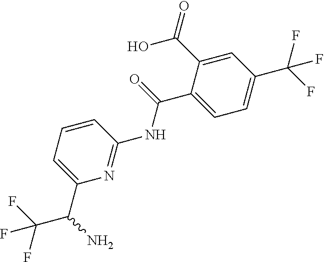 [SMILES: NC(c1cccc(NC(=O)c2ccc(C(F)(F)F)cc2C(=O)O)n1)C(F)(F)F] |
| 22 | 2-({6-[(1R)-1-amino-2,2,2- trifluoroethyl]pyridin-2- yl}carbamoyl)-5- (trifluoromethyl)benzoic acid | 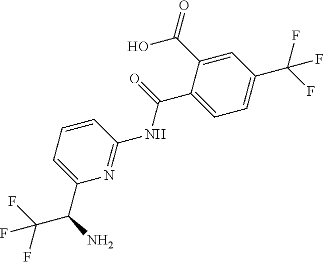 [SMILES: N[C@H](c1cccc(NC(=O)c2ccc(C(F)(F)F)cc2C(=O)O)n1)C(F)(F)F] |
| 23 | 2-({6-[(1S)-1-amino-2,2,2- trifluoroethyl]pyridin-2- yl}carbamoyl)-5- (trifluoromethyl)benzoic acid | 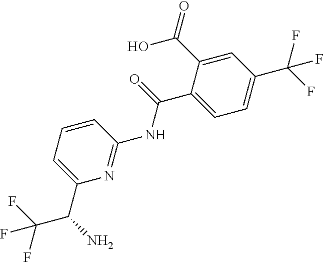 [SMILES: N[C@@H](c1cccc(NC(=O)c2ccc(C(F)(F)F)cc2C(=O)O)n1)C(F)(F)F] |
| 24 | 2-{[6- (methoxymethyl)pyridin-2- yl]carbamoyl}-5- (trifluoromethyl)benzoic acid | 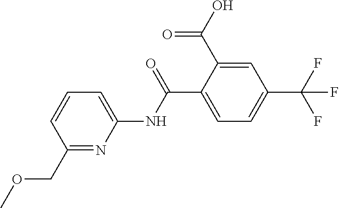 [SMILES: COCc1cccc(NC(=O)c2ccc(C(F)(F)F)cc2C(=O)O)n1] |
| 25 | 5-tert-butyl-2-{[6- (methoxymethyl)pyridin-2- yl]carbamoyl}benzoic acid | 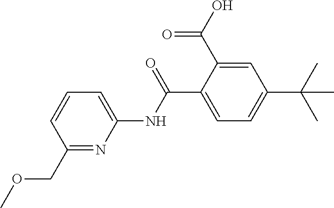 [SMILES: COCc1cccc(NC(=O)c2ccc(C(C)(C)C)cc2C(=O)O)n1] |

### Analytical Methods

### LC-MS

### Method Information

Method 1: LCMS_SC_BASE, Apparatus: Agilent 1260 Bin. Pump: G1312B, degasser; autosampler, ColCom, DAD: Agilent G1315C, 210, 220 and 220-320 nm, PDA 210-320 nm, MSD: Agilent LC/MSD G6130B ESI, pos/neg 100-1000; column: Waters XSelect™ CSH C18, 30×2.1 mm, 3.5 μm, Temp: 25° C., Flow: 1 mL/min, Gradient: t0=5% B, t1.6 min=98% B, t3 min=98% B, Post-time: 1.4 min, Eluent A: 10 mM ammonium bicarbonate in water (pH=9.0), Eluent B: acetonitrile.

Method 2: UPLC_SC_BASE, Apparatus: Waters IClass; Bin. Pump: UPIBSM, SM: UPISMFTN with SO; UPCMA, PDA: UPPDATC, 210-320 nm, SQD: ACQ-SQD2 ESI; ELSD: gas pressure 40 psi, drift tube temp: 50° C., column: Waters XSelect CSH C18, 50'2.1 mm, 2.5 μm, Temp: 25° C., Flow: 0.6 mL/min, Gradient: t0=5% B, t1.3 min=98% B, t1.7 min=98% B, Post-time: 0.3 min, Eluent A: 10 mM ammonium bicarbonate in water (pH=9.5), Eluent B: acetonitrile.

Method 3: UPLC_AN_BASE, Apparatus: Waters IClass; Bin. Pump: UPIBSM, SM: UPISMFTN with SO; UPCMA, PDA: UPPDATC, 210-320 nm, SQD: ACQ-SQD2 ESI; ELSD: gas pressure 40 psi, drift tube temp: 50° C.; column: Waters XSelect CSH C18, 50×2.1 mm, 2.5 μm, Temp: 25° C., Flow: 0.6 mL/min, Gradient: t0=5% B, t2.0 min=98% B, t2.7 min=98% B, Post-time: 0.3 min, Eluent A: 10 mM ammonium bicarbonate in water (pH=9.5), Eluent B: acetonitrile.

Method 4: PREP_ACID-AS3A, Apparatus: Agilent Technologies G6130B Quadrupole; HPLC instrument type: Agilent Technologies 1290 preparative LC; Column: Waters XSelect CSH (C18, 100×30 mm, 10 μ), Flow: 55 ml/min, Column temp: RT; Eluent A: 0.1° /o formic acid in water; Eluent B: 100% acetonitrile; lin. gradient: t=0 min 10% B, t=2 min 10% B, t=8.5 min 50% B, t=10 min 100% B, t=10 min 100% B; Detection: DAD (220-320 nm); Detection: MSD (ESI pos/neg) mass range: 100-1000; fraction collection based on MS and DAD.

Method 5: PREP_ACID-AS4A, Apparatus: Agilent Technologies G6130B Quadrupole; HPLC instrument type: Agilent Technologies 1290 preparative LC; Column: Waters XSelect CSH (C18, 100×30 mm, 10 μ), Flow: 55 ml/min, Column temp: RT; Eluent A: 0.1% formic acid in water; Eluent B: 100% acetonitrile lin. gradient: t=0 min 20% B, t=2 min 20% B, t=8.5 min 60% B, t=10 min 100% B, t=13 min 100% B; Detection: DAD (220-320 nm); Detection: MSD (ESI pos/neg) mass range: 100-1000; fraction collection based on MS and DAD.

### ¹H NMR

1H-NMR spectra were recorded at 400 MHz on a Bruker Avance AV-I-400 instrument or on a Bruker Avance AV-II-400 instrument. Chemical shift values are expressed in ppm-values relative to tetramethylsilane unless noted otherwise. The following abbreviations or their combinations are used for multiplicity of NMR signals: br=broad, d=doublet, m=multiplet, q=quartet, quint=quintet, s=singlet and t=triplet.

### BIOLOGICAL DATA

### Neurotensin Scintillation Proximity Assay

The exemplified compounds of the invention were tested in a Neurotensin (NTS) scintillation proximity assay (SPA) and the IC₅₀ data is shown in Table 2 below. NTS, which is a 13 amino acid neuropeptide, is a sortilin ligand. The IC₅₀ is a measure of the amount of the compound required to inhibit the binding of NTS to sortilin by 50%. The skilled person will recognise that the lower the IC₅₀ value, the less of the compound needed to achieve the desired effect, and as a result, the chances of undesirable off-target effects are reduced.

### Human Sortilin Binding Assay

The compound affinity was determined by measuring the inhibition of ³H-neurotensin and/or ¹²⁵I-neurotensin binding to human full length sortilin using a Scintillation Proximity Assay (SPA) format.

The human sortilin SPA binding assay was performed in a total volume of 40 μl in 50 mM HEPES pH 7.4 assay buffer containing 100 mM NaCl, 2.0 mM CaCl₂, 0.1% (v:v) BSA and 0.1% (v:v)Tween-20. Varying concentration of test compounds where pre-incubated for 30 min at RT with 150 nM of c-terminal hexahistidine- human full length Sortilin (RD Systems). 5 nM [³H]-Neurotensin or 0.35 nM [¹²⁵I]-Neurotensin was added as radioligand and nonspecific binding defined as the binding observed in the presence 20 μM of human Neurotensin. 0.1 mg PVT copper HIS-tag imaging beads (Perkin Elmer) was added and the plate was agitated at 400 rpm in the dark for 1 hr. The SPA beads were allowed a minimum 2 hours settling time before the plate was read on a Hidex Sense 425-301 scintillation reader with a 45 sec exposure time. Concentration-response evaluation of test compounds was performed with 8 different concentrations of compound (covering 3 decades). Using assay high and low controls (0 μM and 20 μM human neurotensin, respectively), relative IC₅₀ values were calculated by nonlinear regression analysis using the sigmoid concentration-response (variable slope) fitting option available in GraphPad Prism 9.0. The compound potency results were converted to inhibition constant Ki values (nM) derived from calculated IC₅₀ values converted to Ki values using the Cheng-Prusoff equation (Ki=IC50/(I+(L/Kd))). Kd for human neurotensin was experimentally determined by saturation binding as 374 nM (n=2).

TABLE 2

| | hSortilin | hSortilin | |
|---|---|---|---|
| | NTS Binding | NTS binding | |
| Example | IC₅₀ (nM) [3H] | IC₅₀ (nM) [125I] | |
| Number | neurotensin | neurotensin | hSortilin Ki |
| 2 | 60 | 105 | |
| 3 | 340 | | 340 |
| 24 | 725 | | |

### REFERENCES

1. Tauris, J., et al., Proneurotrophin-3 May Induce Sortilin-Dependent Death In Inner Ear Neurons. Eur J Neuroscience (2020), 33(4), pp.622-31.
2. Goettsch, C., et al., Sortilin and Its Multiple Roles in Cardiovascular and Metabolic Diseases. Atherosclerosis, Thrombosis and Vascular Biology (2017), 38(1), pp. 19-25.
3. Willnow, T. E., et al., Sortilins: new players in lipoprotein metabolism. Current Opinion in Lipidology (2011), 22(2), pp. 79-85.
4. Kjolby, M., et al., Sort1, encoded by the cardiovascular risk locus 1p13.3, is a regulator of hepatic lipoprotein export. Cell Metabolism (2010), 12(3), pp. 213-223.
5. Jansen, P., et al., Roles for the pro-neurotrophin receptor sortilin in neuronal development, aging and brain injury. Nature Neuroscience (2007), 10(11), pp.1449-1457.
6. Tenk, H. K., et al., ProBDNF induces neuronal apoptosis via activation of a receptor complex of p75NTR and sortilin. J Neuroscience (2005), 10(11), pp.1449-1457.
7. Nykjaer, A., et al., Sortilin is essential for proNGF-induced neuronal cell death. Nature (2004), 427(6977), pp.843-848.

Sequences referenced throughout the
specification and forming part of the
description.
(full length sortilin-isoform 1)
SEQ ID NO: 1

| | |
|---|---|
| 1 | MERPWGAADG LSRWPHGLGL LLLLQLLPPS |
| | TLSQDRLDAP PPPAAPLPRW |
| 51 | SGPIGVSWGL RAAAAGGAFP RGGRWRRSAP |
| | GEDEECGRVR DFVAKLANNT |
| 101 | HQHVFDDLRG SVSLSWVGDS TGVILVLTTF |
| | HVPLVIMTFG QSKLYRSEDY |
| 151 | GKNFKDITDL INNTFIRTEF GMAIGPENSG |
| | KVVLTAEVSG GSRGGRIFRS |
| 201 | SDFAKNFVQT DLPFHPLTQM MYSPQNSDYL |
| | LALSTENGLW VSKNFGGKWE |
| 251 | EIHKAVCLAK WGSDNTIFFT TYANGSCKAD |
| | LGALELWRTS DLGKSFKTIG |
| 301 | VKIYSFGLGG RFLFASVMAD KDTTRRIHVS |
| | TDQGDTWSMA QLPSVGQEQF |
| 351 | YSILAANDDM VFMHVDEPGD TGFGTIFTSD |
| | DRGIVYSKSL DRHLYTTTGG |
| 401 | ETDFTNVTSL RGVYITSVLS EDNSIQTMIT |
| | FDQGGRWTHL RKPENSECDA |
| 451 | TAKNKNECSL HIHASYSISQ KLNVPMAPLS |
| | EPNAVGIVIA HGSVGDAISV |
| 501 | MVPDVYISDD GGYSWTKMLE GPHYYTILDS |
| | GGIIVAIEHS SRPINVIKFS |
| 551 | TDEGQCWQTY TFTRDPIYFT GLASEPGARS |
| | MNISIWGFTE SFLTSQWVSY |
| 601 | TIDFKDILER NCEEKDYTIW LAHSTDPEDY |
| | EDGCILGYKE QFLRLRKSSM |
| 651 | CQNGRDYVVT KQPSICLCSL EDFLCDFGYY |
| | RPENDSKCVE QPELKGHDLE |
| 701 | FCLYGREEHL TTNGYRKIPG DKCQGGVNPV |
| | REVKDLKKKC TSNFLSPEKQ |
| 751 | NSKSNSVPII LAIVGLMLVT VVAGVLIVKK |
| | YVCGGRFLVH RYSVLQQHAE |
| 801 | ANGVDGVDAL DTASHTNKSG YHDDSDEDLL |
| | E |

(full length sortilin-isoform 2)
SEQ ID NO: 2

| | |
|---|---|
| 1 | MERPWGAADG LSRWPHGLGL LLLLQLLPPS |
| | TLSQDRLDAP PPPAAPLPRW |
| 51 | SGPIGVSWGL RAAAAGGAFP RGGRWRRSAP |
| | GEDEECGRVR DFVAKLANNT |
| 101 | HQHVFDDLRG SVSLSWVGDS TGVILVLTTF |
| | HVPLVIMTFG QSKLYRSEDY |
| 151 | GKNFKDITDL INNTFIRTEF GMAIGPENSG |
| | KVVLTAEVSG GSRGGRIFRS |
| 201 | SDFAKNFVQT DLPFHPLTQM MYSPQNSDYL |
| | LALSTENGLW VSKNFGGKWE |
| 251 | EIHKAVCLAK WGSDNTIFFT TYANGSCTDL |
| | GALELWRTSD LGKSFKTIGV |
| 301 | KIYSFGLGGR FLFASVMADK DTTRRIHVST |
| | DQGDTWSMAQ LPSVGQEQFY |
| 351 | SILAANDDMV FMHVDEPGDT GFGTIFTSDD |
| | RGIVYSKSLD RHLYTTTGGE |
| 401 | TDFTNVTSLR GVYITSVLSE DNSIQTMITF |
| | DQGGRWTHLR KPENSECDAT |
| 451 | AKNKNECSLH IHASYSISQK LNVPMAPLSE |
| | PNAVGIVIAH GSVGDAISVM |
| 501 | VPDVYISDDG GYSWTKMLEG PHYYTILDSG |
| | GIIVAIEHSS RPINVIKFST |
| 551 | DEGQCWQTYT FTRDPIYFTG LASEPGARSM |
| | NISIWGFTES FLTSQWVSYT |
| 601 | IDFKDILERN CEEKDYTIWL AHSTDPEDYE |
| | DGCILGYKEQ FLRLRKSSVC |
| 651 | QNGRDYVVTK QPSICLCSLE DFLCDFGYYR |
| | PENDSKCVEQ PELKGHDLEF |
| 701 | CLYGREEHLT TNGYRKIPGD KCQGGVNPVR |
| | EVKDLKKKCT SNFLSPEKQN |
| 751 | SKSNSVPIIL AIVGLMLVTV VAGVLIVKKY |
| | VCGGRFLVHR YSVLQQHAEA |
| 801 | NGVDGVDALD TASHTNKSGY HDDSDEDLLE |

(mature sortilin)
SEQ ID NO: 3

| | |
|---|---|
| 1 | MTFGQSKLYR SEDYGKNFKD ITDLINNTFI |
| | RTEFGMAIGP ENSGKVVLTA |
| 51 | EVSGGSRGGR IFRSSDFAKN FVQTDLPFHP |
| | LTQMMYSPQN SDYLLALSTE |
| 101 | NGLWVSKNFG GKWEEIHKAV CLAKWGSDNT |
| | IFFTTYANGS CTDLGALELW |
| 151 | RTSDLGKSFK TIGVKIYSFG LGGRFLFASV |
| | MADKDTTRRI HVSTDQGDTW |
| 201 | SMAQLPSVGQ EQFYSILAAN DDMVFMHVDE |
| | PGDTGFGTIF TSDDRGIVYS |
| 251 | KSLDRHLYTT TGGETDFTNV TSLRGVYITS |
| | VLSEDNSIQT MITFDQGGRW |
| 301 | THLRKPENSE CDATAKNKNE CSLHIHASYS |
| | ISQKLNVPMA PLSEPNAVGI |
| 361 | VIAHGSVGDA ISVMVPDVYI SDDGGYSWTK |
| | MLEGPHYYTI LDSGGIIVAI |
| 401 | EHSSRPINVI KFSTDEGQCW QTYTFTRDPI |
| | YFTGLASEPG ARSMNISIWG |
| 451 | FTESFLTSQW VSYTIDFKDI LERNCEEKDY |
| | TIWLAHSTDP EDYEDGCILG |
| 501 | YKEQFLRLRK SSVCONGRDY VVTKQPSICL |
| | CSLEDFLCDF GYYRPENDSK |
| 551 | CVEQPELKGH DLEFCLYGRE EHLTTNGYRK |
| | IPGDKCQGGV NPVREVKDLK |
| 601 | KKCTSNFLSP EKQNSKSNSV PIILAIVGLM |
| | LVTVVAGVLI VKKYVCGGRF |
| 651 | LVHRYSVLQQ HAEANGVDGV DALDTASHTN |
| | KSGYHDDSDE DLLE |

1. A compound of formula (I) 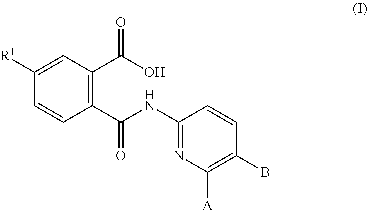
or a pharmaceutically acceptable salt, solvate, hydrate, tautomer, optical isomer, N-oxide, and/or prodrug thereof, wherein
A is selected from the group consisting of H, —OH, C₁-C₄ hydroxyalkyl, C₂-C₄ alkyl, C₁-C₄ haloalkyl, C₁-C₄ alkoxy, C₁-C₄ haloalkoxy and C₁-C₄ aminoalkyl;
B is selected from the group consisting of H, —OH, C₁-C₄ alkyl, C₁-C₄ haloalkyl, C₁-C₄ alkoxy and C₁-C₄ haloalkoxy; or
A and B may be taken together with the carbon atom to which they are respectively attached to form a 5- to 7-membered monocyclic heterocyclic ring that is optionally substituted with one or more substituents independently selected from —OH, C₁-C₄ alkyl, C₁-C₄ haloalkyl and C₁-C₄ alkoxy and C₁-C₄ haloalkoxy; and
R¹ is selected from the group consisting of halo, C₁-C₆ alkyl, C₂-C₆ alkenyl, halo C₁-C₆ alkyl, and halo C₂-C₆ alkenyl.

2. The compound according to claim 1, wherein R¹ is selected from the group consisting of halo, C₁-C₄ alkyl and halo C₁-C₄ alkyl, preferably fluoro C₁-C₄ alkyl.

3. The compound according to claim 1, wherein R¹ is selected from the group consisting of CF₃ and t-butyl.

4. The compound according to any prcccding claim 1, wherein A is selected from the group consisting of C₁-C₄ hydroxyalkyl and C₁-C₄ alkoxy.

5. The compound according to claim 1, wherein B is selected from the group consisting of H, C₁-C₄ haloalkyl and C₁-C₄ alkyl, preferably H.

6. The compound according to claim 1, wherein A and B are taken together with the carbon atom to which they are respectively attached to form a 5- to 7-membered monocyclic heterocyclic ring that is optionally substituted with one or more substituents independently selected from C₁-C₄ alkyl and C₁-C₄ haloalkyl.

7. The compound according to claim 6, wherein A and B are taken together with the carbon atom to which they are respectively attached to form a 6-membered monocyclic heterocyclic ring that is optionally substituted with one or more substituents independently selected from C₁-C₄ alkyl and C₁-C₄ haloalkyl, preferably the 6-membered monocyclic heterocyclic ring is unsubstituted.

8. The compound according to claim 7, wherein the 6-membered monocyclic heterocyclic ring comprises an oxygen atom.

9. The compound according to claim 1, wherein the compound of Formula (I) is
2-({5H,6H,8H-pyrano[3,4-b]pyridin-2-yl}carbamoyl)-5-(trifluoromethyl)benzoic acid;
2-{[6-(hydroxymethyl)pyridin-2-yl]carbamoyl}-5-(trifluoromethyl)benzoic acid;
5-tert-butyl-2-{[6-(hydroxymethyl)pyridin-2-yl]carbamoyl}benzoic acid;
(RAC) 2-{[6-(1-hydroxyethyl)pyridin-2-yl]carbamoyl}-5-(trifluoromethyl)benzoic acid;
2-({6-[(1S)-1-hydroxyethyl]pyridin-2-yl}carbamoyl)-5-(trifluoromethyl)benzoic acid;
2-({6-[(1R)-1-hydroxyethyl]pyridin-2-yl}carbamoyl)-5-(trifluoromethyl)benzoic acid;
2-{[6-(2-hydroxypropan-2-yl)pyridin-2-yl]carbamoyl}-5-(trifluoromethyl)benzoic acid;
2-{[6-(2-hydroxyethyl)pyridin-2-yl]carbamoyl}-5-(trifluoromethyl)benzoic acid;
(RAC) 5-tert-butyl-2-{[6-(1-hydroxyethyl)pyridin-2-yl]carbamoyl}benzoic acid;
5-tert-butyl-2-({6-[(1R)-1-hydroxyethyl]pyridin-2-yl}carbamoyl)benzoic acid;
5-tert-butyl-2-({6-[(1S)-1-hydroxyethyl]pyridin-2-yl}carbamoyl)benzoic acid;
2-({2H,3H,4H-pyrano[2,3-b]pyridin-7-yl}carbamoyl)-5-(trifluoromethyl)benzoic acid;
2-({2H,3H-[1,4]dioxino[2,3-b]pyridin-6-yl}carbamoyl)-5-(trifluoromethyl)benzoic acid;
2-({5H,7H,8H-pyrano[4,3-b]pyridin-2-yl}carbamoyl)-5-(trifluoromethyl)benzoic acid;
2-[(5,6,7,8-tetrahydro-1,6-naphthyridin-2-yl)carbamoyl]-5-(trifluoromethyl)benzoic acid;
5-tert-butyl-2-({5H,6H,8H-pyrano[3,4-b]pyridin-2-yl}carbamoyl)benzoic acid;
(RAC) 2-{[6-(2,2,2-trifluoro-1-hydroxyethyl)pyridin-2-yl]carbamoyl}-5-(trifluoromethyl)benzoic acid;
2-({6-[(1S)-2,2,2-trifluoro-1-hydroxyethyl]pyridin-2-yl}carbamoyl)-5-(trifluoromethyl)benzoic acid;
2-({6-[(1R)-2,2,2-trifluoro-1-hydroxyethyl]pyridin-2-yl}carbamoyl)-5-(trifluoromethyl)benzoic acid;
(RAC) 2-{[6-(1-amino-2,2,2-trifluoroethyl)pyridin-2-yl]carbamoyl}-5-(trifluorom ethyl)benzoic acid;
2-({6-[(1R)-1-amino-2,2,2-trifluoroethyl]pyridin-2-yl}carbamoyl)-5-(trifluoromethyl)benzoic acid;
2-({6-[(1S)-1-amino-2,2,2-trifluoroethyl]pyridin-2-yl}carbamoyl)-5-(trifluoromethyl)benzoic acid;
or a pharmaceutically acceptable salt, solvate, hydrate, tautomer, optical isomer, N-oxide, and/or prodrug thereof.

10. A pharmaceutical composition comprising a compound according to claim 1 and a pharmaceutically acceptable carrier, excipient, and/or diluent.

11. (canceled)

12. A method of treating or preventing a neurodegenerative disorder, a cancer, pain, diabetes mellitus, diabetic retinopathy, cardiovascular disease, hereditary eye conditions or hearing loss comprising administering the pharmaceutical composition of claim 10 to a subject.

13. The method of claim 12, wherein the neurodegenerative disorder is selected from frontotemporal dementia, Alzheimer's disease, Parkinson's disease and spinal cord injury.

14. The method of claim 12, wherein the cancer is selected from breast cancer, lung cancer, ovarian cancer, prostate cancer, thyroid cancer, pancreatic cancer, glioblastoma and colorectal cancer.

15. The method of claim 12, wherein the hearing loss is selected from noise-induced hearing loss, ototoxicity induced hearing loss, age-induced hearing loss, idiopathic hearing loss, tinnitus and sudden hearing loss.
